(19) United States
(12) Patent Application Publication
(10) Pub. No.: US 2018/0185297 A1
(43) Pub. Date: Jul. 5, 2018

(54) Serum Specific Drug Transport System

(71) Applicants: Daniel DeBrouse, Walnut Creek, CA (US); Terrence Rockwell, Walnut Creek, CA (US)

(72) Inventors: Daniel DeBrouse, Walnut Creek, CA (US); Terrence Rockwell, Walnut Creek, CA (US)

(21) Appl. No.: 15/860,555

(22) Filed: Jan. 2, 2018

Related U.S. Application Data
(60) Provisional application No. 62/441,766, filed on Jan. 3, 2017.

(51) Int. Cl. A61K 9/50; A61K 9/00; A61K 9/06; A61K 38/28

(52) U.S. Cl. CPC A61K 9/5036; A61K 9/0053; A61K 38/28; A61K 9/06; A61K 9/5068

(57) ABSTRACT

An oral transenterocytotic mucosal adhesion vehicle includes a ghost cell formed from fermented bacterium providing a container for uptake and active transport via a lacteal surface and a nano encapsulated drug particle including a bioactive agent which is encapsulated by a polymeric coating disposed within said ghost cell and surviving first pass liver metabolism, wherein the polymeric coating comprises molecules of an alginate and transmucosal delivery enhancing molecules, wherein the transmucosal delivery enhancing molecules are covalently conjugated to the alginate molecules, wherein the polymeric delivery vehicle is resistant to intestinal degradation and wherein the polymeric delivery vehicle is capable of transmucosal passage across the intestinal mucosa into the lymphatic capillary wherein the polymeric delivery vehicle comprising the alginate molecules and transmucosal delivery enhancing molecules covalently conjugated thereto is degraded to release substantially all of the bioactive agent.

### BACKGROUND OF INVENTION

### 1. Field of Invention

The presently claimed and disclosed inventive relates generally to pharmaceutical and nutriceutical products, and more particularly to improved novel transmucosal delivery vehicle for delivery of pharmaceutical and nutriceutical bioactive agents via plasma membrane stimulants, such as phospholipids, to target enterocyte receptors for entry by receptor mediated endocytosis and methods of their production and methods of their use.

### 2. Prior Art

Orally consumed pharmaceutical or nutriceutical bioactive material have recently been delivered and absorbed into the bloodstream through the wall of the small intestine or large intestine using different techniques. Enteric coatings have been used to encapsulate oral dosage forms to prevent damage to the active substance contained in the oral preparation by acids and enzymes in the stomach. Such coatings permit the active substance to pass to the small intestine, where upon reaching, the active substance is released. Enteric coatings provide a delivery approach, although less desirable the other methods.

The lumen of the small intestine and the blood vasculature of the intestinal mucosa have been identified as ideal dissolution targets for a wide variety of bioactive pharmaceutical and nutriceutical compounds. Thus, the focus recently is in providing a vehicle which is able to permeate through the intestinal wall.

The instant inventor has provided serum specific nano encapsulation or “SSNe” for short as a transport vehicle wherein any drug may be fitted for its subsequent disposition in the mammalian blood stream in the absence of those effects drugs are exposed to as a result of first pass metabolism. The vehicle is resistant to gastrointestinal disintegration and is taken up by enterocytes lining the wall of the small intestine. The endocytic mechanism at play is an example of an active transport whereby the SSNe vehicle binds to a transport protein present in the plasma membrane of enterocytes as a transmembrane species. This binding initiates the active movement of the SSNe vehicle from the lumen of the intestine into the cytoplasm of a cell. Once in the cell the SSNe vehicle is processed along the same pathway as cholesterol and is dumped in the lacteal. Noteworthy is the SSNe vehicle interacts and was designed to interact with an isoprenoid receptor. The SSNe vehicle is the first serum specific drug delivery system ever devised.

While this delivery system significantly improved the state-of-the-art, there remains a need to improve technology. The instant invention provides significant improvements in the state-of-the-art.

### SUMMARY OF THE INVENTION

It is an object to improve serum specific drug transport technology.

It is another object to provide a serum specific truck transport vehicle which uses peptide technology to stimulate plasma membrane of a cell and targets enterocyte receptors for cytosolic entry buy receptor mediated endocytosis.

It is a third object of the invention to provide a phospholipid transport based vehicle.

Another object is to provide unique transenterocytotic mucosal adhesion vehicle delivery.

Accordingly, one embodiment of invention is directed to a serum specific drug transport vehicle which uses peptide “antigens” to target highly concentrated enterocyte receptors for cytosolic entry by receptor mediated endocytosis to simulate the plasma membrane of a cell. The transmembrane targets are ones that are highly specialized and evolved for the active transport of certain biomolecules across the phospholipid bilayer.

Another aspect of the invention is drifting to another serum specificity which uses phospholipids in place of the instant inventor's prior alginate backbone structure. This sets a selective permeability which may be used by the clinician in early to mid to late stage post-oral administration. For example, such vehicles may incorporate a drug cocktail whose therapeutics may be dropped off in separate points within the lymphatic and systemic circulation. In this embodiment, the serum specific drug transport technology, phospholipids such as phosphatidylethanolamine, phosphatidylcholine, phosphatidylserine or similar or a combination thereof can be is used in place of the alginate polymer. An exemplary embodiment provides at least two phospholipids brought together to form a bilayer membrane around a therapeutic agent held within the vehicle core.

A ghost cell membrane, preferably a Lactobacillus “ghost” cell membrane is provided for containment of the serum specific drug transport vehicle. This provides for a unique transenterocytotic mucosal adhesion vehicle delivery.

### BRIEF DESCRIPTION OF THE DRAWINGS

FIG. 1 depicts a serum specific drug transport vehicle having at least two phospholipids brought together to form a bilayer membrane around a therapeutic agent held within the bilayer membrane.

FIG. 2 depicts a serum specific drug transport vehicle having at least two phospholipids brought together to form a bilayer membrane enclosure around a therapeutic agent held within a vehicle core.

FIG. 3 depicts a serum specific drug transport vehicle having an outer phospholipid layer spherically about an inner phospholipid layer wherein a therapeutic agent held between the hydrophilic tails of the phospholipids.

FIG. 4 depicts a bilayer sheet of a serum specific drug transport vehicle of the invention.

FIG. 5 depicts a lactobacillus acidophilus ghost cell.

FIG. 6 depicts a transenterocytotic mucosal adhesion vehicle delivery across bilayer membrane.

FIG. 7 depicts the sodium glucose transport enterocyte transit.

FIG. 8 depicts transenterocytotic mucosal adhesion vehicle adsorption and transport.

FIG. 9 depicts nano encapsulation of a drug.

FIG. 10 depicts transenterocytotic mucosal adhesion vehicle endocytosis comparison.

FIG. 11 depicts a model of a transenterocytotic mucosal adhesion vehicle of the invention.

### DETAILED DESCRIPTION OF THE PREFERRED EMBODIMENTS

Referring now to the Figures, a serum specific drug transport vehicle is generally represented by the numeral 10, 20, 40 and 50. In the referring to FIG. 1, the serum specific drug transport vehicle 10 has an outer membrane 12 or shell of which includes a plurality of phospholipids 14 in the form of a bilayer with selective permeability with hydrophilic tails 16 of the phospholipids 14 adjacent one another and having delivery antigen molecule 18 selectively placed throughout the formed membrane 12.

Antigen molecule 18 is covalently linked between phospholipids 14 to one location, but it is understood the location can be any desired possible location present in the membrane 12. It is this linkage, antigen molecule 18 will bind to a transmembrane protein of an enterocyte known to be involved in the active transport of a biomolecule. For example, one target is a sodium-glucose linked transporter, SGLT family of glucose transporters found in the intestinal mucosa (enterocytes) of the small intestine (SGLT1). In this instance, antigen molecule 18 will include a glucose residue covalently linked to a backbone formed of phospholipid 14 in membrane 12 of the transport vehicle.

The serum specific drug transport vehicle 10 in some cases trends upwards to an absolute bioavailability's of 100% of the administered dose. It is recognized that a small fraction of the dose would normally pass to and through one's large intestine going out as waste. Thus, the instant invention contemplates that an absolute bioavailability approaches 95% of administered dose in humans though 100% is contemplated in human as well as animal subjects.

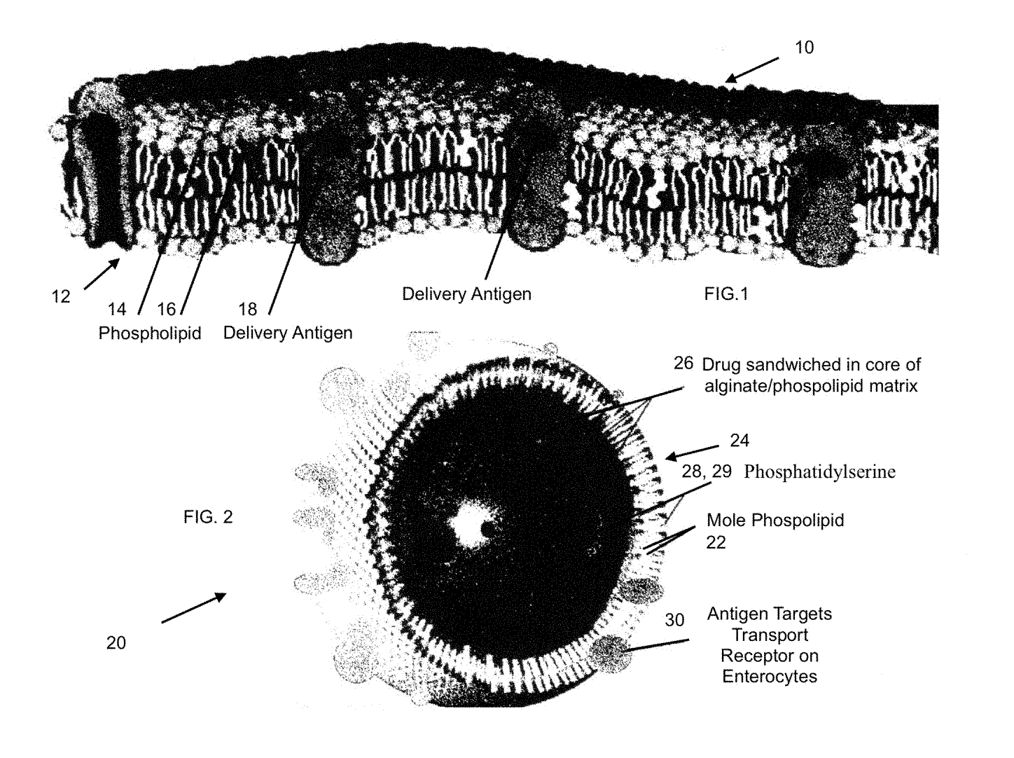

FIG. 2 provides depicts a serum specific drug transport vehicle 20 having at least two phospholipids 22 brought together to form a bilayer membrane 24 enclosure around a therapeutic agent 26 held within a vehicle core 28. Here, therapeutic agent (drug) 26 is sandwiched in the core 28 of an alginate/phospholipid matrix molecule 29. Antigen 26 targets transport receptor 30 on enterocytes.

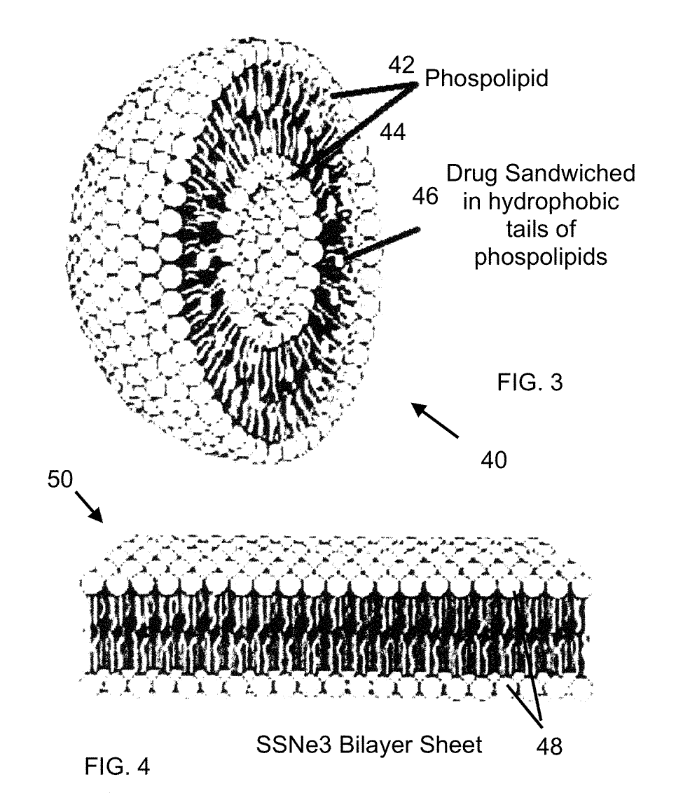

FIG. 3 depicts a serum specific drug transport vehicle 40 having an outer phospholipid layer 42 spherically about an inner phospholipid layer 44. A therapeutic agent 46 is held between hydrophilic tails 46 of the phospholipid layers 42 and 44. FIG. 4 depicts a bilayer sheet 48 of a serum specific drug transport vehicle 50 of the invention.

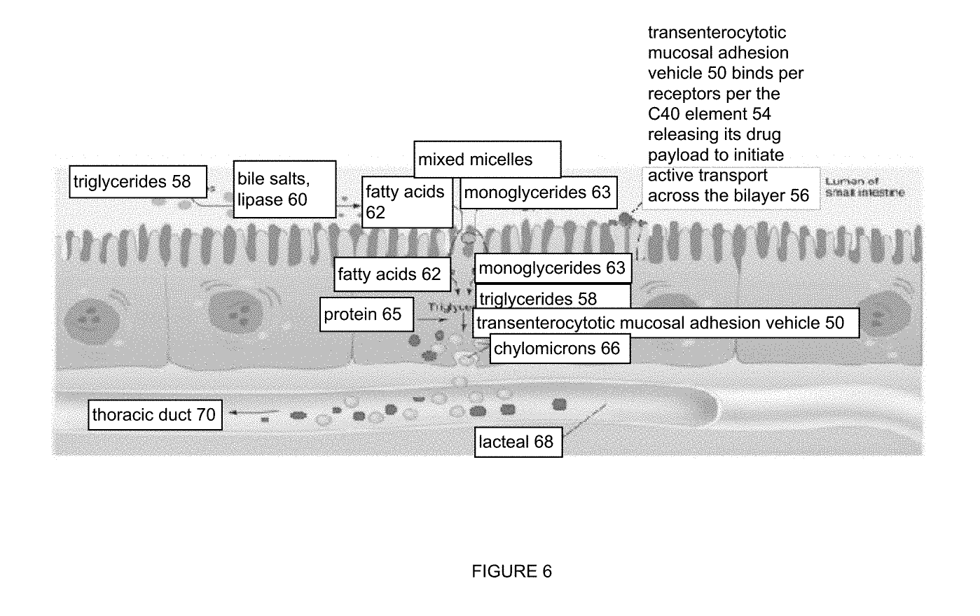

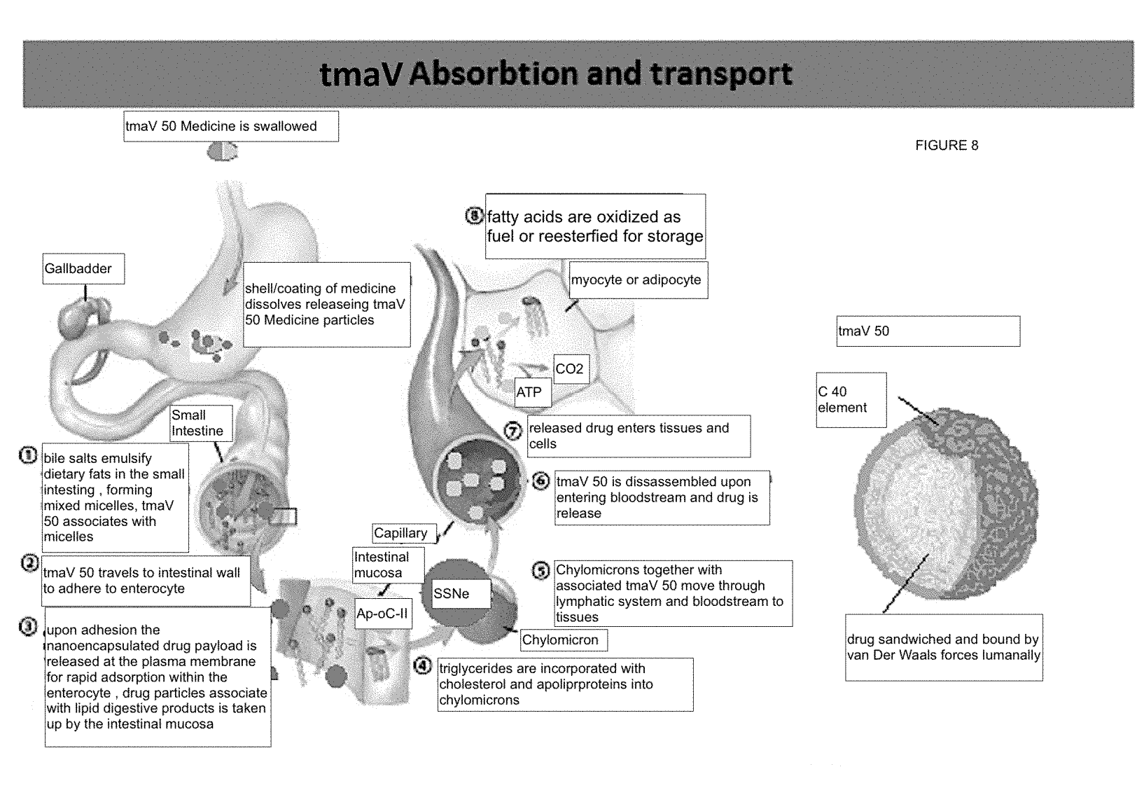

FIG. 6 depicts another diagram of operation of transenterocytotic mucosal adhesion vehicle 50. Here, there is depicted a lumen of small intestine 52 wherein the transenterocytotic mucosal adhesion vehicle 50 binds per receptors per the C40 element 54 releasing its drug payload to initiate active transport across the bilayer 56. Triglycerides 58, bile salts and lipase 60, fatty acids 62, monoglycerides 63 with mixed micelles 64 and these along with protein 65 and transenterocytotic mucosal adhesion vehicle 50 provide for release of chylomicrons 66 through lacteal 68 into thoracic duct 70. Upon oral administration of a pill or capsule containing the transenterocytotic mucosal adhesion vehicle 50 (the formation of which is described hereinafter) enters a gastric fluid, and releases the therapeutic payload. The transenterocytotic mucosal adhesion vehicle 50 is 100% resistant to the gastrointestinal tract and will travel with digestive micelles into the intestinal mucosa where it adheres with a high affinity the enterocyte bilayer 56 for drug payload disposition through lacteal 68. The lymphatic capillary will move transenterocytotic mucosal adhesion vehicle 50 drug away from the major sites of first pass metabolism eventually dumping them into the blood where the vehicles will disassemble a predetermined period (e.g. 15 minutes) post dumping to release clinically viable drug. FIG. 8 depicts the transenterocytotic mucosal adhesion vehicle 50 adsorption and transport path through the body.

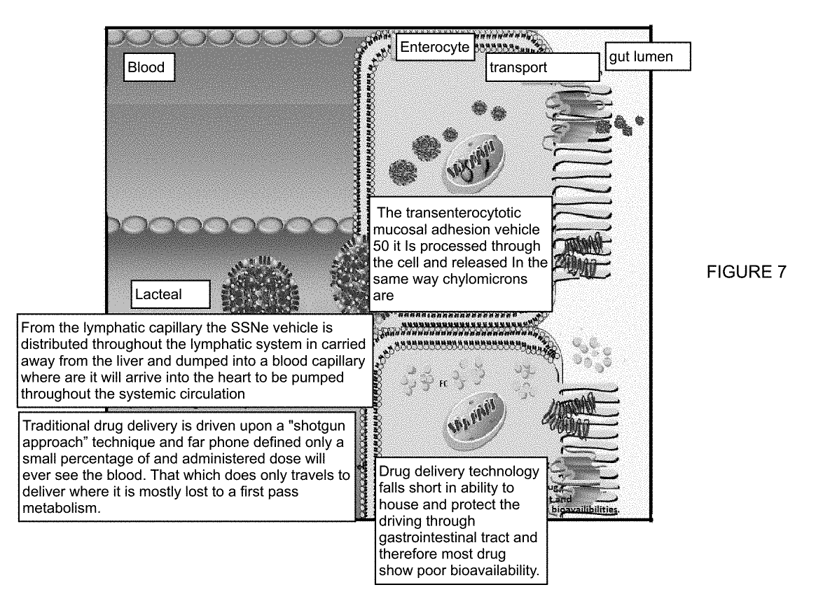

FIG. 7 depicts the sodium glucose transport enterocyte transit. As illustrated here, transenterocytotic mucosal adhesion vehicle 50 gains entrance through endocytosis involving its active transport across the living bilayer 56 (see FIG. 6). The transenterocytotic mucosal adhesion vehicle 50 it Is processed through the cell and released in the same way chylomicrons are. From the lymphatic capillary the SSNe vehicle is distributed throughout the lymphatic system in carried away from the liver and dumped into a blood capillary where are it will arrive into the heart to be pumped throughout the systemic circulation. Traditional drug delivery is driven upon a “shotgun approach” technique and far phone defined only a small percentage of and administered dose will ever see the blood. That which does only travels to deliver where it is mostly lost to a first pass metabolism. Drug delivery technology falls short in ability to house and protect the driving through gastrointestinal tract and therefore most drug show poor bioavailability.

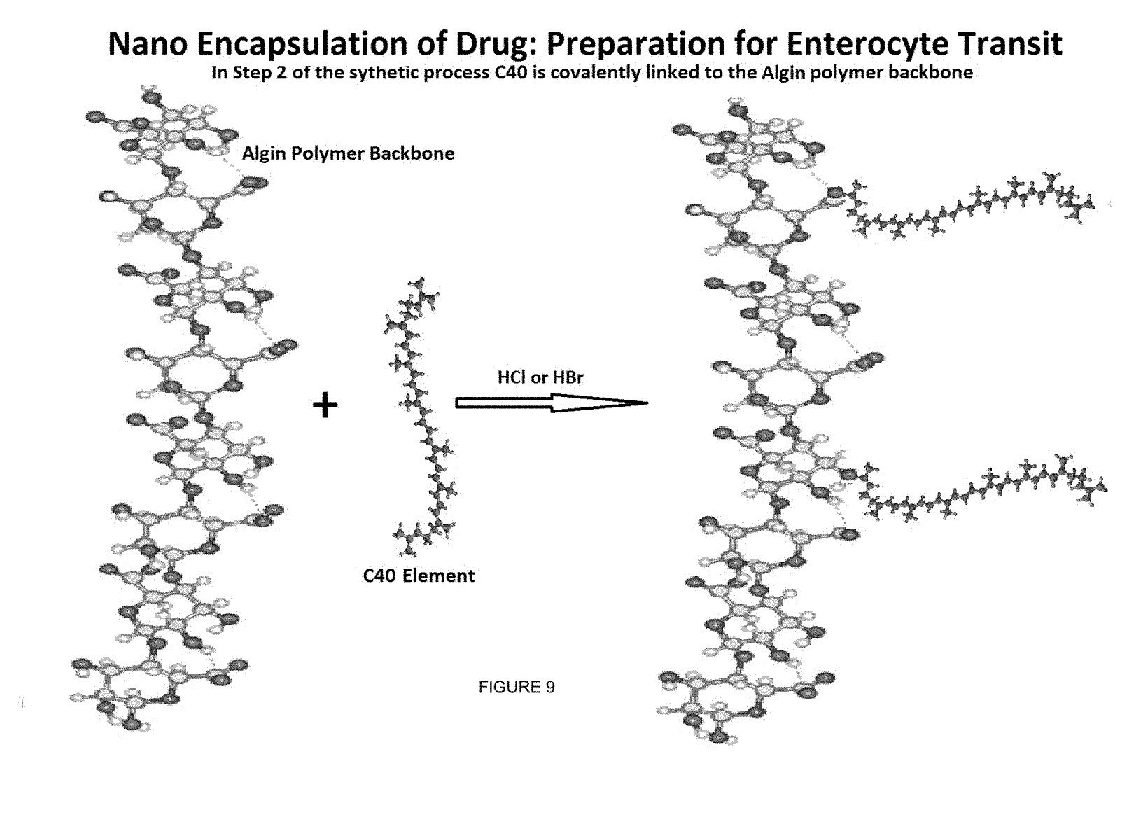

FIG. 9 depicts an example of nano encapsulation of drug by preparation for enterocyte transit. Here, an algin polymer backbone combines with a C 40 element by way on a synthetic process covalently linking to the polymer.

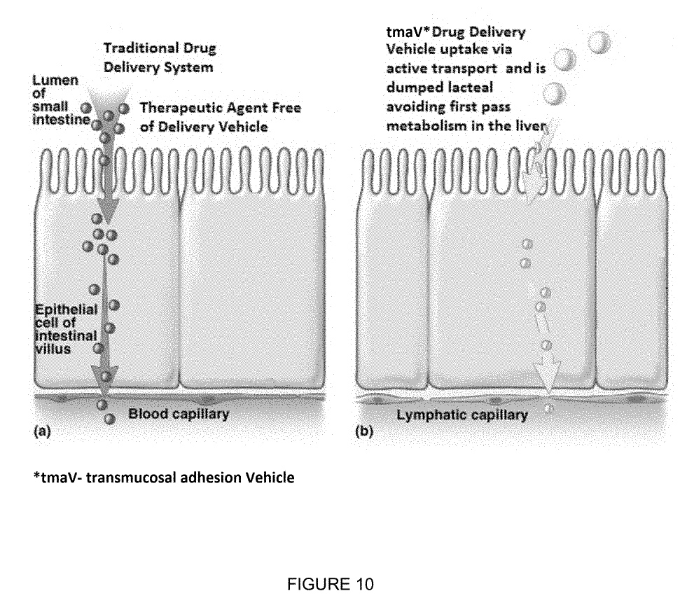

FIG. 10 depicts transenterocytotic mucosal adhesion vehicle endocytosis comparison. Part (a) shows the traditional drug delivery system via the lumen of the small intestine in through the blood capillary. Part (b) shows the transenterocytotic mucosal adhesion vehicle uptake via Active transport and which is dumbed lacteal avoiding first pass metabolism in the lymphatic capillary.

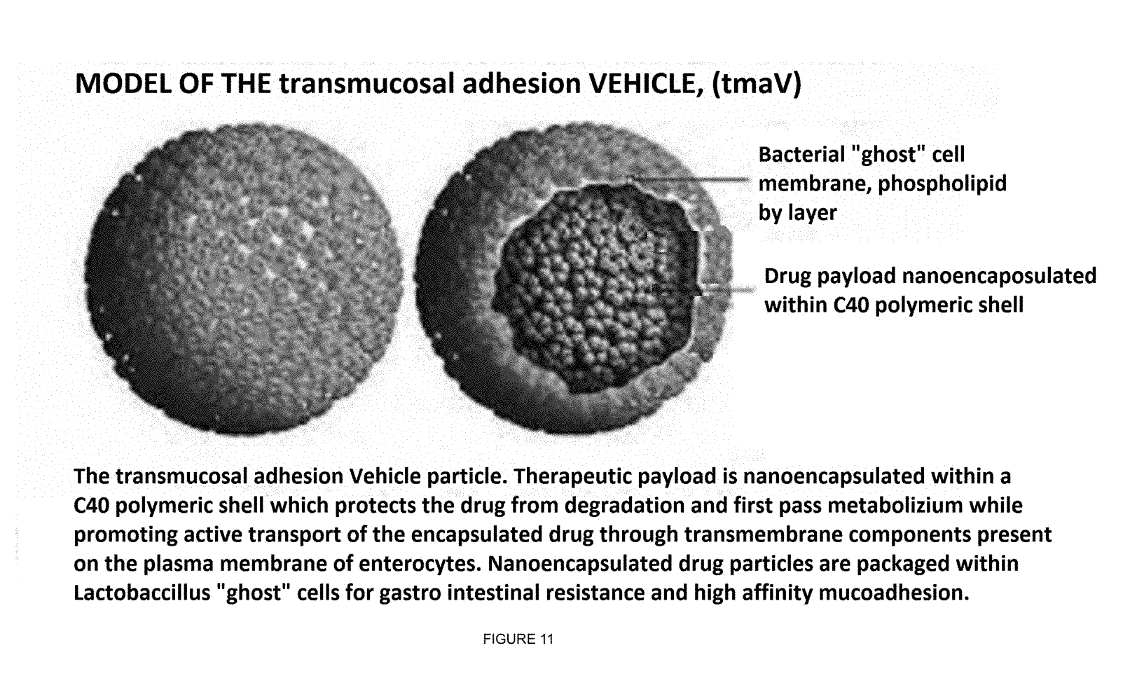

FIG. 11 depicts transenterocytotic mucosal adhesion vehicle model. The trans mucosal adhesion vehicle particle here provides for a ghost cell membrane, phospholipid by layer. A drug payload nano encapsulated within C 40 polymeric shell contained within the ghost cell membrane protects the drug from the degradation in first pass metabolism while promoting active transport of the encapsulated drug through the transmembrane components present on the plasma membrane the enterocytes. Nano encapsulated drug particles are packaged within Lactobacillus “ghost” cells for gastrointestinal resistance and high infinity mucoadhesion.

A method of encapsulation is provided as follows.

Mixed-Strain Starter Cultures.

L. acidophilus strains NRRL B-1910 and B-2092 were used for fermentation. Lyophilized cultures were first cultivated in deep liver medium and then transferred to soybean milk medium. Each culture was incubated at 37° C. for 24 hours and was serially transferred at least twice in the same medium before using as a starter. These cultures were grown singly and blended in the ratio of 1:1 just before their addition to soybean milk medium for fermentation.

Soybean Milk Medium

5% sucrose was added to the soybean milk. Sucrose-enriched soybean milk. However. stirring slow in titratable acidity lifter 24 hours at 57° C. in a mixed culture fermentation (Table 31. To stimulate bacterial growth which, in turn, would increase titratable acidity. Cheddar cheese whey solids were added to the milk, as whey solids increased from 0 to 4%.

The biomass of the 72 hr cultivated L. acidophilus culture was collected by centrifuging the bacterial broth at 4000 rpm for 10 min. The cells then washed gently with 0.5% saline and re-centrifuged at 4000 rpm for 10 min. The supernatant was then discarded. The Lactobacilli cells were pelletized and washed. The cell concentration was equal to 106×10 to the tenth power CFU/m L. 5× concentration stock for each of NaOH, SDS, and H202 were prepared from both of the +1 and −1 values, which were determined from the MIC and the MGC as above. Twelve experiments were conducted following the design of the Plackett-Burman as in Table 1. Either −1 or +1 value of each variable was used according to the design. All the experiments were conducted in three steps. The first step contains all the variables except H202. After saline/water washing, the second step contains only H202. In the first step one ml of each of the 5× (+1 or −1) NaOH, SDS, and CaCO3 was added to 2 mL of the bacterial suspension to have a final volume equal to 5 mL and to give a final concentration equal to 1× for each. After 1 hr incubation according to the Plackett-Burman design, the different mixtures (for each experiment) were centrifuged each and the cells were collected as cell pellets using centrifugation at 4000 rpm for 10 min. The supernatant was then transferred to clean and sterile Falcon tubes.

The cell pellets were then washed with 0.5% sterile saline and re-centrifuged. The supernatant then discarded and the cells suspended in 2 ml of the + or − value of H202 according to Plackett-Burman design for 30?min. The cells and the supernatant were collected each as above. The cell pellets then washed by saline solution Followed by centrifugation (as above). Finally, in the third step, the cell pellets were resuspended in 60% Ethanol and left at room temperature for 30 min with gentle vortexing every 5 min for 30 sec. The cell pellets collection and washing were repeated as above.

TABLE 1
Plackett - Burman experimental design.
Exp. no. Variables Basic Experiment V Basic Experiment H202

| H202 Ethanol | Ethanol | Response | | Shaking rate/ | |
|---|---|---|---|---|---|
| SDS | H202 | CaC03 | NaOH | Temperature | DNA μg/mL |
| Protein μg/mL | DNA μg/mL | Protein μg/mL | DNA μg/mL | Protein μg/mL | B6Q % |
| 1 | 1 | 1 | 1 | −1 | 1 |
| 164.5 | 3425.445 | 20.2 | 353.547 8.1 | 45.477 | 100 |
| 2 | −1 | −1 | −1 | −1 | −1 |
| 136.2 | 2400.012 | 16.45 | 432.765 | 11.5 500.247 | 90 |
| 3 | 1 | −1 | 1 | 1 | −1 |
| 178.6 | 3137.913 | 0.60 | 16.55 | 391.689 | 10 |
| 4 | −1 | −1 | −1 | 1 | 1 |
| 74.2 | 2589.255 | 4.4 | 151.101 | 5.65 123.228 | 30 |
| 5 | −1 | 1 | 1 | 1 | −1 |
| 121.15 | 2078.739 | 19.25 | 337.41 | 38.142 | 50 |
| 6 | 1 | −1 | 1 | −1 | 99.85 |
| 3082.167 | 16.1 | 222.984 | 5.4 | 233.253 | 60 |
| 7 | −1 | 1 | −1 | −1 | −1 |
| 106.1 | 1880.694 | 33 | 651.348 | 4.75 126.162 | 90 |
| 8 | −1 | −1 | 1 | 1 | 1 |
| 146.9 | 2400.012 | 33 | 284.598 | 7.7 29.34 | 0 |
| 9 | 1 | 1 | −1 | 1 | 1 |
| 0 | 0 | 5.15 | 968.22 | 123.6 4.401 | 0 |
| 10 | −1 | 1 | 1 | −1 | 1 |
| 90.15 | 1805.877 | 13.1 | 1043.037 | 6.8 36.675 | 80 |
| 11 | 1 | −1 | −1 | −1 | 1 |
| 122.2 | 2100.744 | 12.05 | 355.014 | 5.05 36.675 | 100 |
| 12 | 1 | −1 | 1 | −1 | −1 |
| 59.4 | 2565.783 | 8.15 | 234.72 | 6.75-365.283 | 60 |

Next bioactives for nano-encapsulation were either 1) encapsulated within calcium alginate shells prior to packaging within Lactobacilli ghosts, or 2) packaged directly into Lactobacilli ghosts or 3) left raw unencapsulated as control.

The human gastrointestinal tract (GIT) harbors a complex symbiotic microbial community. Humans and their symbiotic bacteria have co-evolved and their mutual interactions are essential for human health and well-being. Intestinal bacteria play a key role in modulating development of the host immune system and barrier properties of the intestinal epithelium.

Lactobacilli are critical intestinal symbiotic bacteria to maintaining the intestinal microbial ecosystem and to providing protection against pathogen infection. Lactobacilli are used in the food and fermentation industries for their probiotic capabilities. Lactobacilli species are resistant to harsh environments of the gastrointestinal tract and in the intestine they adhere to and interact with the epithelium and the mucosal layers where nutrient uptake takes place. Lactobacilli adherence to the intestinal epithelium promotes persistence colonization, stimulates immunomodulation, and provides protection per antagonistic activities against pathogens.

Lactobacilli adhere to the intestinal epithelium through bacterial cell surface adhesions, polysaccharides, and proteins which bind individually or collectively to corresponding transmembrane receptors present on intestinal enterocyte membranes. The bindings between bacterium and host enterocytes are interactions which inventor believes might lead to pathogen exclusion and immunomodulation of host cells. The adhesive properties of lactobacilli are directly linked to their surface properties which are influenced by the structure and composition of their cell wall.

There is a great diversity in Lactobacilli cell surface architecture and the bacteria modify their cell surface properties in response to local environmental changes. For example, lactobacilli express cell wall constituent integrity proteins during environmental stress which preserve the cell wall. The cell surface architecture of lactobacilli and their ability to express certain surface components, or to secrete specific factors which act directly on host cells, influence the physicochemical properties of the bacterial cell and lead to strain-specific properties.

Presented is a method by which Lactobacilli may be cultivated under conditions that promote a) structural enhancement of the cell wall, b) increased ligand expression, c) increased and broadened ligand mucoadhesion through recombinantly engineered ligand modifying adeno viruses and d) Lactobacilli “ghost-cell” production and finally a method by which the “ghost-cells” may be packaged with nano-spheres of a single therapeutic agent or a cocktail of therapeutic agents for subsequent incorporation into oral dosage form for patient administration.

Two vector classes will be demonstrated and evaluated separately. In addition, it is preferred to employ only wild type methodologies for each phase of the manufacturing process as outlined above and no external chemistry shall be applied. The method brings together the appropriate population of organisms and nutrients of natural origin capable of carrying out each phase of vehicle production.

The Transenterocytotic Mucosal Adhesion Vehicle

### “Ghost-Lactobacilli”

In 2012 after some 10 years of research by the inventor, Dr. Daniel DeBrouse, this culminated this into US Patent Application 20100226995 for his development of the first drug delivery vehicle presenting 100% resistant to gastrointestinal disintegration, an isomeric variety of surface ligands bound to a polymeric alginate backbone which forms a tightly wound sphere of alginate double-helical aggregates cross-linked and sandwiching drug in between its helical configuration. DeBrouse devised the ligand component for high affinity interactions with 3 or more transmembrane proteins involved in the active transport of isoprenoids across the enterocyte bilayer for disposition into the lymph vessels. In this manner nano packages of therapeutic agents are successfully delivered to the blood sera by route of the lymph vessels, arriving first to the heart and pumped throughout all tissues avoiding first pass metabolism. The nano spheres of encapsulated therapeutic agent remain intact from oral administration until their arrival to the blood sera and tissues where disassembly and drug release occurs 15 minutes post serum disposition. The array of therapeutic agents and classes thereof which may be successfully doped is broad in spectrum including proteins, peptides, DNA, RNA and small molecules, whether water or lipid soluble. From the start it was DeBrouse's objective to simulate a cellular membrane or viral capsid capable of precision drug dispositions direct within blood sera of defined mechanism at bio adsorption rates comparable to those of intravenous infusion. With this generation one technology DeBrouse has introduced an entire new field of drug delivery science and shown how biochemical symbiosis and mimicry may be applied in the development of and administration of precision therapeutics.

Transmucosal delivery enhancing molecules contemplate at least one of an isoprenoid compound, a vitamin, a signal peptide, or a fatty acid having 6-28 carbon atoms. Further, examples of transmucosal delivery enhancing molecules comprise at least one of lycopene, limonene, gamma-tocotrienol, geraniol, carvone, farnesol, geranylgeraniol, squalene or other linear terpenoids, a carotenoid, taxol, vitamin E, vitamin A, beta-carotene, Coenzyme Q10 (ubiquinone), astaxanthin, zeaxanthin, lutein, citranxanthin, beta-choro-carotene, and canthroaxanthan.

The delivery vehicle can further include at least one of a gum, a gum resin, a resin, glycerin, high fructose corn syrup, and a fruit or vegetable juice, cellulose gums, pectins, pectin resins, locust bean gums, locust bean resins, xanthan gums, xanthan gum resins, carrageenans, sodium salts of carrageenans, gellan gums, gellan gum resins, whey protein gums, whey protein resins, agar agar, propylene glycol, Arabic gums, Arabic gum resins, guar gum, guar gum resins, gum tragacanth, and gum ghatti.

The delivery vehicle contemplates use of an aqueous base including water, and at least one of glycerin, a surfactant, or propylene glycol or an oil base which comprises at least one of soybean oil, peanut oil, sesame oil, safflower oil, canola oil, cotton seed oil, olive oil, corn oil, and/or vegetable oil. The delivery vehicle of the aqueous or oil base can include between about <1% to 80% of the composition by weight.

The delivery vehicle contemplates an absorbent factor including at least one of glycyrrhizinate, glycrrhetinic acid, sucrose fatty acid ester, glycerin, glycerol fatty acid ester, adipic acid, polyethylene glycol, sodium dodecyl sulfate, sodium caprate, and sodium deoxycholate, sodium chloride, potassium chloride, calcium chloride or any combination thereof.

The delivery vehicle also contemplates that the bioactive agent comprises at least one of an antibiotic, an antiviral agent, a protease inhibitor, a polypeptide, a chemotherapeutic agent, an anti-tumor agent, an anti-sense drug, insulin, an RNA, a DNA, an immunosuppressant, a vaccine, a protein, a microorganism, a peptidomimetic, or nutriceutical.

The transmucosal delivery enhancing molecules include <0.5% to 30% of the vehicle by weight. The pH modulator and/or protease inhibitor includes <0.5% to 10% of the vehicle by weight. The delivery vehicle of the instant invention includes a ghost cell in the size of 1 nm to 10 μm in diameter. The delivery vehicle has a gel consistency. The polymeric delivery vehicle has a solid consistency.

In early 2015 the inventor began reconsidering this approach realizing that a more desirable and more precise drug transport system would be one in which the vehicles enterocyte specificity, affinity and surface interaction would be assured even absolute through the vehicles dependence upon a enterocyte mucoadhesive mechanism. Such a system contemplated enhancing the absolute bioavailability of a drug to levels equal to those achieved upon its intravenous administration. This could result in mucoadhesive-dependent drug disposition which would outperform intravenous administrations revealing routine bio availabilities in excess of 99% of an administered dose. Furthermore, mucoadhesive-dependent drug disposition will revolutionize extended release therapeutics by allowing precision release in relation to time post administration and even providing a means by which a payload of therapeutics could be housed at the enterocyte surface and released at specific chemically programmed intervals of time even extending a 30-day period.

Lactobacilli acidophilus ghost cell is provided for the preparation of a transenterocytotic mucosal adhesion vehicle (TMAV). It has been found that this ghost cell provides superior enterocyte mucous adhesion affinity in comparison to other intestinal flora. Further, due to its harmless colonization within the small intestine and it is existing Generally Recognized As Safe “GRAS” status within the United States FDA. It is also a widely recognized probiotic.

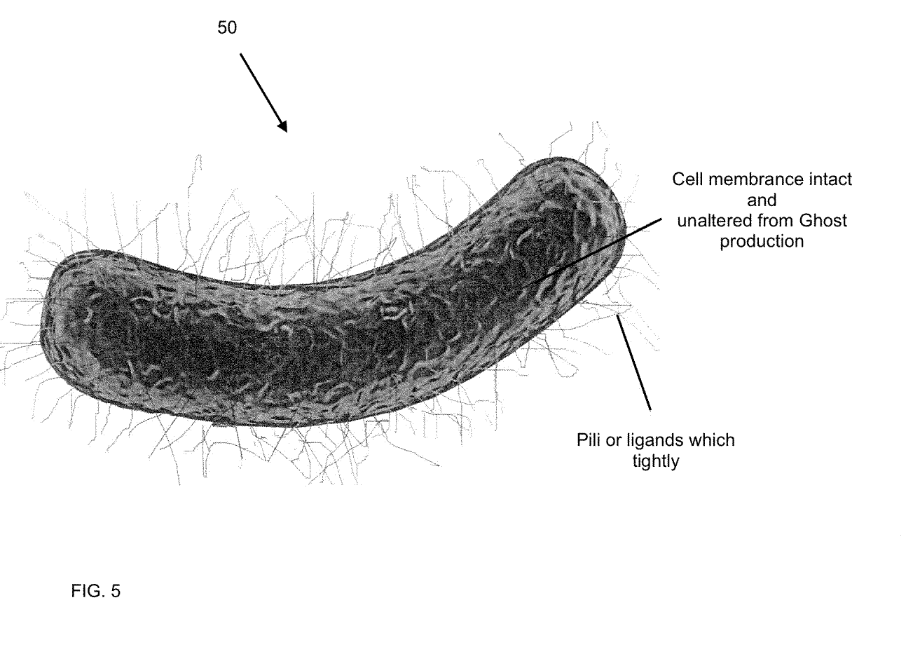

FIG. 5 depicts a lactobacillus acidophilus ghost cell 50 which has pili or ligands too tightly bind enterocytes. Transenterocytoytic Mucosal Adhesion Vehicles are prepared by the removal of all intracellular constituents followed by the electroporation packaging of drug. Lactobacillus acidophilus is a member of the Lactobacillus genus of bacteria. These bacteria can be found in the mouth, intestine, and vagina. L. acidophilus benefits health through its production of vitamin K and lactase. This bacteria is commonly used in food, such as yogurt, dairy products, and fermented soy products, such as miso and tempeh.

The bacterium is fermented in a feed batch system. Upon harvesting the bacterial cake it is rehydrated in an aqueous pH buffered solution and treated with sonication and electroporation-like technique which favors the opening of voltage gated ion channels as follows.

L. acidophilus NCC2628 was obtained from the Nestle Culture Collection. Cultures were grown at 40° C. under anaerobic conditions in test tubes containing 10 ml of medium and harvested in late stationary phase (12 to 14 h). A number of media, designated M1, M2, M3, and M4, were used. The complete medium was M1, which consisted of 1.00% Variolac 836 (MD Foods Ingredients, Denmark) by weight, 0.10% Tween 80 (Quest International, The Netherlands) by weight, 0.75% sucrose (Fluka, Switzerland) by weight, 0.75% fructose (Fluka, Switzerland) by weight, 1.00% Pisane (Cosucra, France) by weight, 3.00% yeast extract 2012 (Biospringer, France) by weight, and 1.00% Primatone RL (Quest International, The Netherlands) by weight. Variolac 836 is a whey permeate powder consisting largely of lactose but also containing about 4% proteins. Pisane is a pea protein concentrate, whereas Primatone is an enzymatic digest of meat high in amino acids and peptides. M2, M3, and M4 were each lacking one of the essential components of M1. M2 lacked sucrose and lactose, M3 lacked Pisane and Primatone, and M4 lacked the yeast extract. After fermentation, the bacteria were harvested by centrifugation (5,000×g, 10 min, 4° C.) and washed twice with 0.9% sodium chloride solution. The viable cell count was determined by serially diluting 1 ml of homogenized cell culture in 100 mM NaH₂PO₄ buffer. The dilutions were plated on MRS agar and anaerobically incubated for 24 hours at 40° C.

Preparation of L. acidophilus Ghosts

Bacterial ghosts are produced by the controlled expression of ϕX174 lysis gene E. E-mediated lysis of bacteria results in the formation of empty bacterial cell envelopes, which have the same cell surface composition as their living counterparts. They display all surface components in a natural non denatured form. Even highly sensitive and fragile structures like pili are well protected by this instant invention technology.

L. acidophilus Ghosts were produce in a 10 L fermentor (Meredos, Bovenden, Germany) with a stirring rate of 350 rpm and 3.5 liters of air per min. No antifoam was added, and pH values were stable in the range (6.5 to 7.5) necessary for successful mediated lysis. To induce nuclease expression, Isopropyl β-D-1-thiogalactopyranoside (IPTG) was added to the cultures at an OD₆₀₀ of 0.3. Protein mediated lysis was induced 45 min later by a temperature shift from 28° C. to 42° C. MgCl₂ and CaCl₂ were added 90 min after induction of lysis. The number of colony forming unit (CFU) within the bacterial samples taken during lysis and nuclease was determined using the spiral plater. Samples were serially diluted in 0.85% NaCl, inoculated onto LB agar plates, and incubated at 28° C. overnight.

The ghosts were collected by centrifugation 6 hours after lysis induction and washed three times with 0.85% NaCl solution with ⅓, ⅙, and finally 1/12 of the starting culture volume). The final cell pellet was resuspended in 20 ml distilled water and freeze-dried for 24 hours. TMAV were stored at −20° C. until use. Ten milligrams of lyophilized ghost preparations was inoculated in LB, incubated for 1 week 28° C., and analyzed for living-cell counts by plating on LB agar plates.

### Therapeutic Payload

10 mg of the L. acidophilus “ghosts” were suspended in 20 mL of nano pure water and exposed to ultrasonication for a period of 60 min. At 15 post sonication, while continuing to sonicate, 5 mg of resverotrol was slowly added by rocker/shaker directly into the center of the TMAV containing aqueous medium over a period of 15 minutes to post sonication 30 minutes. Sonication was continued until post sonication 60 minutes. Next the TMAV therapeutic vectors were collected by centrifugation and washed 3× with 0.85% sodium chloride solution. The vector pellet was resuspended in nano pure water and lyophilized.

Electroporation of L. acidophilus Ghosts

Species=L. acidophilus

Dilution=1/50

Growth=MRS+1% Glycine, 37° C., 3-4 hours

Wash=5 mM NaH2PO4 1 mM MgCl2 (Ice Cold)

Buffer=0.3M Sucrose 5 mM NaH2PO4 1 mM MgCl2 (Ice Cold)

Concentration=1/100

Voltage=7,000 V/cm

Recovery=90 min MRS

Although lactobacillus ghost cells have preferred muco adhesion

### Preparation of Therapeutic Agent Nanoencapsulation

Part 1.

Electrophilic addition of HCl to C40 33.34 g of C40 isoprenoid was dissolved in acetone and loaded into the reaction Vessel of a HCl gas generation system and HCl gas was flowed directly into the reaction vessel at 25° C. for a period of 30 minutes. At the end of the 30 minute reaction period, the reaction was stopped and acetone was removed from the isoprenoid by distillation at 56.6° C. (+/−2° C.). 200 mg of halogenated isoprenoid was then combined with 20.0 g of AANa in DMSO and sonicated (pulse: 1 sec/2 sec at 60% power) at 4° C. for a period of 1 hr. DMSO was removed by freeze drying and the resulting AANa-C40 was slowly warmed to 25° C. and washed 4 times with actetone, ethanol a Milli-Pore water, respectively. AANa-C40 was then rapidly frozen in liquid nitrogen and freeze dried for a period of 2 hrs. AANa-C40 was stored at −22° C. until use.

Part 2.

Nanoencapsulation of Insulin 0.198 moles of CaCl was dissolved in 10 mL of Milli-Pore water and stirred at 25° C. until dissolved. The solution was then vacuum filtered through a 0.45 um membrane and a placed under a sonication probe. The sonicator was set at a pulse of 1 sec/sec at 77% power for a period of 180 seconds at 4° C. The tip of the probe was placed below the surface, 1.3 cm depth, of the CaCl solution and the sonication cycle was started. 200 mg of AANa-C40 was mixed completely with 50 mg of Insulin and added to the calcium chloride solution at a rate of 9.2 mg/sec. Upon completion of the sanitation cycle the mixture was centrifuged at 3000×g 4° C. for a period of 20 minutes and the supernatant discarded. SSNe particles were washed 4 times with Milli-Pore water and freeze dried for a period of 2 hours and then stored at −22° C. until rodentdminstration. This process yielded 207.3 mg of Insulin loaded SSNe particles 1-2 m in diameter at a concentration of 250 ug/mg.

Electroporation of L. acidophilus Ghosts for Nanoencapsulate Packaging

Species=L. acidophilus

Dilution=1/50

Growth=MRS+1% Glycine, 37° C., 3-4 hours

Wash=5 mM NaH2PO4 1 mM MgCl2 (Ice Cold)

Buffer=0.3M Sucrose 5 mM NaH2PO4 1 mM MgCl2 (Ice Cold)

Concentration=1/100

Voltage=7,000 V/cm

Recovery=90 min MRS

The presently claimed and disclosed inventive concept(s) and its advantages have been described in detail. It should be understood that various changes, modifications and derivations can be made herein without departing from the spirit and scope of the presently claimed and disclosed inventive concept(s) as defined by the appended claims.

The scope of the present application is not intended to be overly limited to the particular embodiments of the process, formulas, compounds, compositions of matter, means, methods, mechanisms and steps described in the specification. As one of ordinary skill in the art will readily appreciate from the disclosure of the presently claimed and disclosed inventive concept(s), processes, formulas, compounds, compositions of matter, means, mechanisms, methods, or steps, presently existing or later to be developed that perform substantially the same function, also those which achieve substantially the same result as the corresponding embodiments described herein may be utilized according to the present invention. Accordingly, the appended claims are intended to include within their scope such processes, formulas, compounds, compositions of matter, means, mechanisms, methods, or steps.

1. An oral transenterocytotic mucosal adhesion vehicle, comprising:
a ghost cell formed from fermented bacterium providing a container for uptake and active transport via a lacteal surface; and
a nano encapsulated drug particle including a bioactive agent which is encapsulated by a polymeric coating disposed within said ghost cell and surviving first pass liver metabolism, wherein the polymeric coating comprises molecules of an alginate and transmucosal delivery enhancing molecules, wherein the transmucosal delivery enhancing molecules are covalently conjugated to the alginate molecules, wherein the polymeric delivery vehicle is resistant to intestinal degradation and wherein the polymeric delivery vehicle is capable of transmucosal passage across the intestinal mucosa into the lymphatic capillary wherein the polymeric delivery vehicle comprising the alginate molecules and transmucosal delivery enhancing molecules covalently conjugated thereto is degraded to release substantially all of the bioactive agent.

2. The oral transenterocytotic mucosal adhesion vehicle of claim 1, wherein the ghost cell is formed from L. acidophilus bacterium.

3. The oral transenterocytotic mucosal adhesion vehicle of claim 1, wherein the alginate is sodium alginate, potassium alginate, and/or calcium alginate.

4. The oral transenterocytotic mucosal adhesion vehicle of claim 1, wherein the alginate molecules are cross-linked.

5. The oral transenterocytotic mucosal adhesion vehicle of claim 1, wherein said nano encapsulated drug particle includes one of a pharmaceutical and nutriceutical bioactive agent.

5. The oral transenterocytotic mucosal adhesion vehicle of claim 1, wherein the transmucosal delivery enhancing molecules comprise at least one of an isoprenoid compound, a vitamin, a signal peptide, or a fatty acid having 6-28 carbon atoms.

6. The oral transenterocytotic mucosal adhesion vehicle of claim 1, wherein the transmucosal delivery enhancing molecules comprise at least one of lycopene, limonene, gamma-tocotrienol, geraniol, carvone, farnesol, geranylgeraniol, squalene or other linear terpenoids, a carotenoid, taxol, vitamin E, vitamin A, beta-carotene, Coenzyme Q10 (ubiquinone), astaxanthin, zeaxanthin, lutein, citranxanthin, beta-choro-carotene, and canthroaxanthan.

6. The oral transenterocytotic mucosal adhesion vehicle of claim 1, further comprising at least one of a gum, a gum resin, a resin, glycerin, high fructose corn syrup, and a fruit or vegetable juice.

7. The oral transenterocytotic mucosal adhesion vehicle of claim 1, comprising at least one of the group comprising cellulose gums, pectins, pectin resins, locust bean gums, locust bean resins, xanthan gums, xanthan gum resins, carrageenans, sodium salts of carrageenans, gellan gums, gellan gum resins, whey protein gums, whey protein resins, agar agar, propylene glycol, Arabic gums, Arabic gum resins, guar gum, guar gum resins, gum tragacanth, and gum ghatti.

8. The oral transenterocytotic mucosal adhesion vehicle of claim 1, wherein the aqueous base comprises water, and at least one of glycerin, a surfactant, or propylene glycol.

9. The oral transenterocytotic mucosal adhesion vehicle of claim 1, wherein the oil base comprises at least one of soybean oil, peanut oil, sesame oil, safflower oil, canola oil, cotton seed oil, olive oil, corn oil, and/or vegetable oil.

10. The oral transenterocytotic mucosal adhesion vehicle of claim 1, wherein the absorbent factor comprises at least one of glycyrrhizinate, glycrrhetinic acid, sucrose fatty acid ester, glycerin, glycerol fatty acid ester, adipic acid, polyethylene glycol, sodium dodecyl sulfate, sodium caprate, and sodium deoxycholate, sodium chloride, potassium chloride, calcium chloride or any combination thereof.

11. The oral transenterocytotic mucosal adhesion vehicle of claim 1, wherein the bioactive agent comprises at least one of an antibiotic, an antiviral agent, a protease inhibitor, a polypeptide, a chemotherapeutic agent, an anti-tumor agent, an anti-sense drug, insulin, an RNA, a DNA, an immunosuppressant, a vaccine, a protein, a microorganism, a peptidomimetic, or nutriceutical.

12. The oral transenterocytotic mucosal adhesion vehicle of claim 1, wherein the aqueous or oil base comprises <1% to 80% of the composition by weight.

13. The oral transenterocytotic mucosal adhesion vehicle of claim 1, wherein the transmucosal delivery enhancing molecules comprise <0.5% to 30% of the vehicle by weight.

14. The oral transenterocytotic mucosal adhesion vehicle according to claim 1, wherein the pH modulator and/or protease inhibitor comprises <0.5% to 10% of the vehicle by weight.

15. The oral transenterocytotic mucosal adhesion vehicle of claim 1, wherein the polymeric coating ranges in the size of 1 nm to 10 μm in diameter.

16. The oral transenterocytotic mucosal adhesion vehicle of claim 1, wherein the polymeric delivery vehicle has a gel consistency.

17. The oral transenterocytotic mucosal adhesion vehicle of claim 1, wherein the polymeric delivery vehicle has a solid consistency.
